(19) United States
(12) Patent Application Publication
(10) Pub. No.: US 2022/0157448 A1
(43) Pub. Date: May 19, 2022

(54) DETECTION OF ABNORMAL CHANGES IN FLOW PULSATILITY CLINICALLY CORRELATED WITH ADVERSE EVENTS USING LOGFILE DATA

(71) Applicant: HeartWare, Inc., Miami Lakes, FL (US)

(72) Inventor: Neethu Lekshmi Jalaja Vasudevan, Miami Lakes, FL (US)

(21) Appl. No.: 17/097,169

(22) Filed: Nov. 13, 2020

(51) Int. Cl. G16H 40/67; A61M 1/12; A61M 1/10; A61B 8/06; G16H 15/00; G06F 17/18

(52) U.S. Cl. CPC G16H 40/67; A61M 1/122; G06F 17/18; A61B 8/06; G16H 15/00; A61M 1/1086

(57) ABSTRACT

A method of determining adverse events from log file data from operation of an implantable blood pump. The method includes measuring a flow pulsatility from flow rate data recorded in a log file. A moving mean of the measured flow pulsatility is calculated. A moving standard deviation of the measured flow pulsatility is calculated. A difference between the moving mean of the measured pulsatility and the moving standard deviation of the measured flow pulsatility is calculated. If the difference is less than a first predetermined threshold, an alert is generated as the presence of an adverse event.

### CROSS-REFERENCE TO RELATED APPLICATION

n/a.

### FIELD

The present technology is generally related to a method of determining adverse events from log file data from operation of an implantable blood pump.

### BACKGROUND

Implantable blood pumps, and in particular, ventricular assist devices (VADS), are equipped with controllers configured to log parameters of the operation of the blood pump. For example, in one configuration, certain controllers are configured to log power consumption and flow rate, but does not provide information about abnormal pulsatility. Indeed, many adverse events may be directly correlated with sudden and abnormal changes in pulsatility that last for a certain period of time.

### SUMMARY

The techniques of this disclosure generally relate to a method of determining adverse events from log file data from operation of an implantable blood pump.

In one aspect, the present disclosure provides a method of determining adverse events from log file data from operation of an implantable blood pump. The method includes measuring a flow pulsatility from flow rate data recorded in a log file. A moving mean of the measured flow pulsatility is calculated. A moving standard deviation of the measured flow pulsatility is calculated. A difference between the moving mean of the measured pulsatility and the moving standard deviation of the measured flow pulsatility is calculated. If the difference is less than a first predetermined threshold, an alert is generated as the presence of an adverse event.

In another aspect of this embodiment, the method further includes calculating a difference between consecutive points of the moving standard deviation.

In another aspect of this embodiment, if the difference between consecutive points of the moving standard deviation is less than a second predetermined threshold, the method further includes generating the alert as to the presence of the adverse event.

In another aspect of this embodiment, the method further includes displaying the difference between consecutive points of the moving standard deviation.

In another aspect of this embodiment, the method further includes displaying the moving standard deviation in the log file.

In another aspect of this embodiment, the method further includes displaying the moving mean in the log file.

In another aspect of this embodiment, the method further includes displaying the alert in the log file.

In another aspect of this embodiment, the first predetermined threshold is less than a 1000.

In another aspect of this embodiment, the second predetermined threshold is −180.

In another aspect of this embodiment, the implantable blood pump is a ventricular assist device.

In one aspect, a controller for an implantable blood pump includes processing circuitry configured to: measure a flow pulsatility from flow rate data recorded in a log file; calculate a moving mean of the measured flow pulsatility; calculate a moving standard deviation of the measured flow pulsatility; calculate a difference between the moving mean of the measured pulsatility and the moving standard deviation of the measured flow pulsatility; and if the difference is less than a first predetermined threshold, generate an alert as the presence of an adverse event.

In another aspect of this embodiment, the processing circuitry is further configured to calculate a difference between consecutive points of the moving standard deviation.

In another aspect of this embodiment, if the difference between consecutive points of the moving standard deviation is less than a second predetermined threshold, the processing circuitry is further configured to generate the alert as to the presence of the adverse event.

In another aspect of this embodiment, the processing circuitry is further configured to display the difference between consecutive points of the moving standard deviation.

In another aspect of this embodiment, the processing circuitry is further configured to display the moving standard deviation in the log file.

In another aspect of this embodiment, the processing circuitry is further configured to display the moving mean in the log file.

In another aspect of this embodiment, the processing circuitry is further configured to display the alert in the log file.

In another aspect of this embodiment, the first predetermined threshold is less than 1000.

In another aspect of this embodiment, the second predetermined threshold is −180.

In one aspect, a method of determining adverse events from operation of an implantable blood pump includes measuring a flow pulsatility from flow rate data. A moving mean of the measured flow pulsatility is calculated. A moving standard deviation of the measured flow pulsatility is calculated. A difference between the moving mean of the measured pulsatility and the moving standard deviation of the measured flow pulsatility is calculated. A difference between consecutive points of the moving standard deviation is calculated. If the difference is less than a first predetermined threshold, an alert is generated as the presence of an adverse event. If the difference between consecutive points of the moving standard deviation is less than a second predetermined threshold, the alert is generated as to the presence of the adverse event.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is a block diagram of an exemplary system for an exemplary implantable blood pump;

FIG. 2 is a flow chart of an exemplary method of the present application;

FIG. 3A is an exemplary log file showing features of the present application;

FIG. 3B is a continuation of the log file shown in FIG. 3A;

FIG. 4A is another exemplary log file showing features of the present application; and

FIG. 4B is a continuation of the log file shown in FIG. 4A.

### DETAILED DESCRIPTION

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

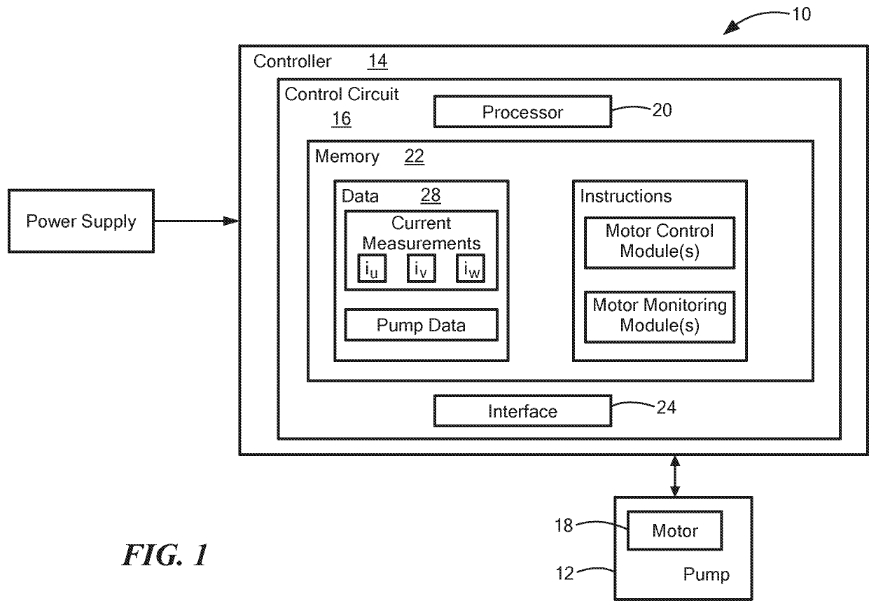

FIG. 1 is a block diagram of the system 10 including an implantable blood pump 12 in communication with a controller 14. The blood pump 12 may be the HVAD® Pump or another mechanical circulatory support device fully or partially implanted within the patient and having a movable element, such as a rotor, configured to pump blood from the heart to the rest of the body. The controller 14 includes a control circuit 16 for monitoring and controlling startup and subsequent operation of a motor 18 implanted within the blood pump 12. The controller 14 may also include a processor 20, a memory 22, and an interface 24. The memory 22 is configured to store information accessible by the processor 20 including instructions 26 executable by the processor 20 and/or data 28 that may be retrieved, manipulated, and/or stored by the processor 20. In particular, the processor 20 includes circuitry configured to carry out the steps discussed herein with respect to the methods. As such, reference to the system 10 executing steps of the methods is intended to include the processor 20.

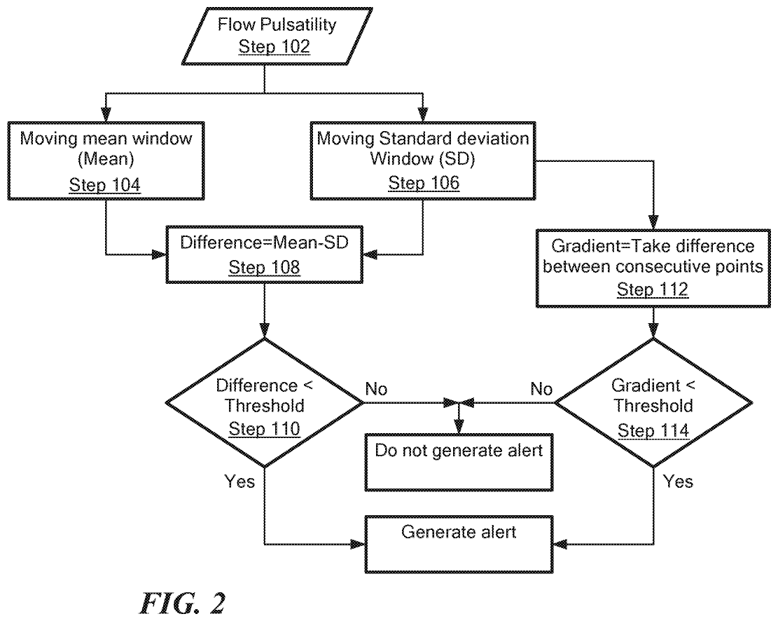

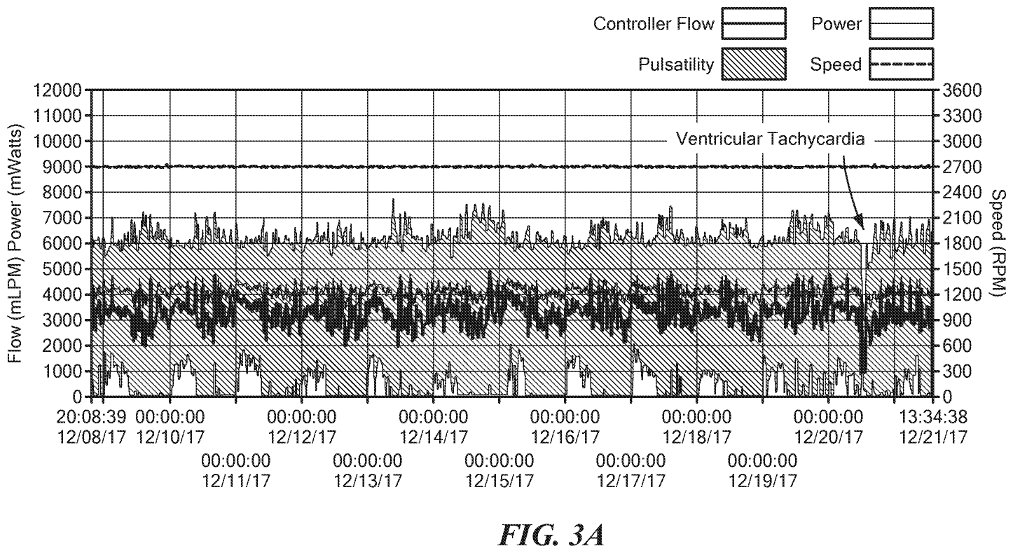

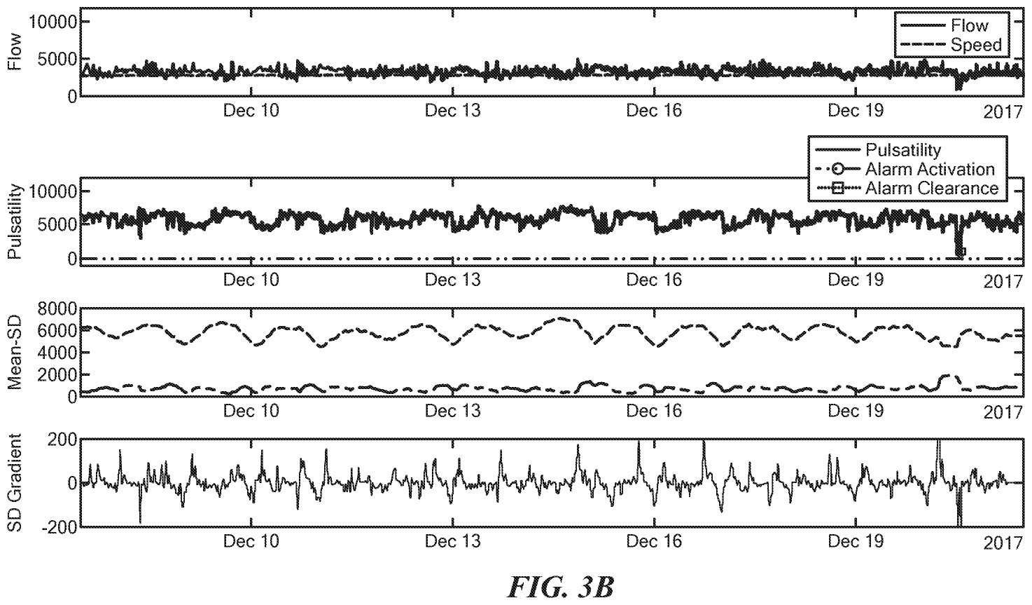

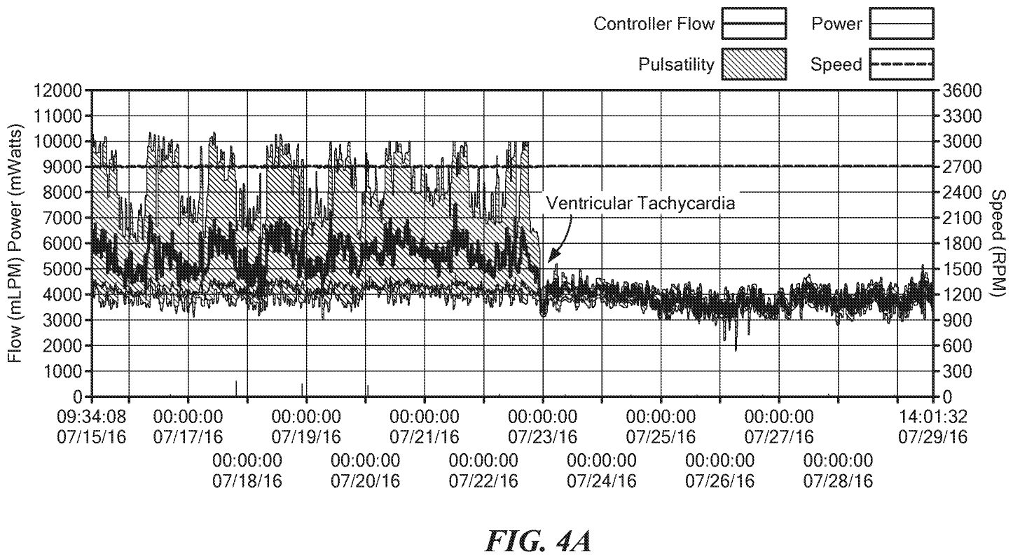

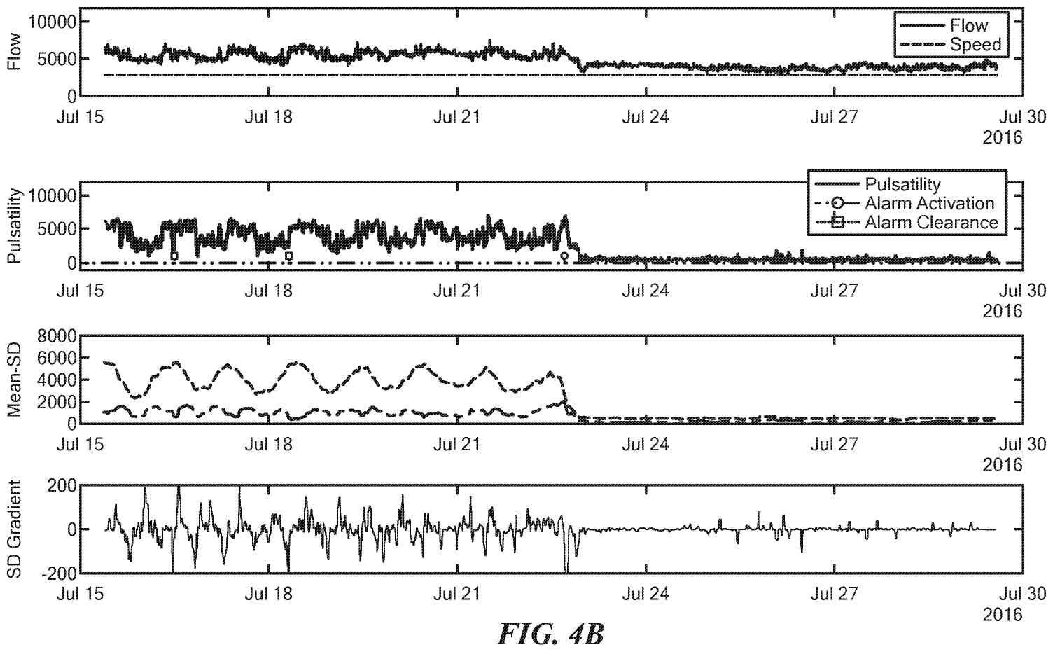

Referring now to FIGS. 2-4, in which an exemplary method of determining adverse events from log file data from operation of the implantable blood pump 12 is shown. In particular, data 28 may be recorded in a log file 30 (exemplary log files are shown in FIGS. 3A, 3B-4A, 4B) from which a variety of pump parameters may be extracted. In an exemplary method, a flow pulsatility is measured from flow rate data recorded in the log file 30 (Step 102). The flow pulsatility may be displayed in the log file 30 as along with other parameters such as a power and RPM speed of an impeller of the blood pump 12. Alternatively, flow pulsatility may be extracted from the log file 30 and displayed by itself. A moving mean of the measured flow pulsatility is calculated (Step 104). The moving mean may be displayed in the log file 30 in a separate window. Moreover, a moving standard deviation of the measured flow pulsatility is calculated (Step 106). The moving standard deviation may be displayed in the same window as the moving mean and may further be displayed in a different color than the moving mean. A difference between the moving mean of the measured pulsatility and the moving standard deviation of the measured flow pulsatility is calculated (Step 108). If this calculated difference is less than a first predetermined threshold, an alert is generated as to the presence of an adverse event, for example ventricular tachycardia (Step 110). In one configuration, the first predetermined threshold is less than 1000. The alert may be a visual alert within the log file 30, or alternatively, may be an audible, tactile, or visual alert generated by the controller 14 when the alert is generated. Moreover, the controller 14 is further configured to calculate a difference between consecutive points of the moving standard deviation (Step 112). For example, the time frame for the log file 30, may be, for example, every 15 minutes. Thus, consecutive data points may be every 15 minutes, or a different time frame. If the difference between consecutive points of the moving standard deviation is less than a second predetermined threshold, the controller 14 is further configured to generate the alert as to the presence of the adverse event (Step 114). The second predetermined threshold in one configuration is −180. The difference between consecutive points of the moving standard deviation may also be displayed in the log file 30 in a separate window.

Although described herein as the controller 14 being configured to log pulsatility information into the log file 30, it is contemplated that the method and the controller 14 may be utilized for real time alerts. For example, the controller 14 may be configured to monitor pulsatility in real-time and generate real-time alerts in accordance with the methods described herein.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed is:

1. A method of determining adverse events from log file data from operation of an implantable blood pump, comprising:
measuring a flow pulsatility from flow rate data recorded in a log file;
calculating a moving mean of the measured flow pulsatility;
calculating a moving standard deviation of the measured flow pulsatility;
calculating a difference between the moving mean of the measured pulsatility and the moving standard deviation of the measured flow pulsatility; and
if the difference is less than a first predetermined threshold, generating an alert as the presence of an adverse event.

2. The method of claim 1, further comprising calculating a difference between consecutive points of the moving standard deviation.

3. The method of claim 2, wherein if the difference between consecutive points of the moving standard deviation is less than a second predetermined threshold, the method further includes generating the alert as to the presence of the adverse event.

4. The method of claim 2, further including displaying the difference between consecutive points of the moving standard deviation.

5. The method of claim 1, further including displaying the moving standard deviation in the log file.

6. The method of claim 1, further including displaying the moving mean in the log file.

7. The method of claim 1, further including displaying the alert in the log file.

8. The method of claim 1, wherein the first predetermined threshold is less than a 1000.

9. The method of claim 1, wherein the second predetermined threshold is −180.

10. The method of claim 1, wherein the implantable blood pump is a ventricular assist device.

11. A controller for an implantable blood pump, comprising:
processing circuitry configured to:
measure a flow pulsatility from flow rate data recorded in a log file;
calculate a moving mean of the measured flow pulsatility;
calculate a moving standard deviation of the measured flow pulsatility;
calculate a difference between the moving mean of the measured pulsatility and the moving standard deviation of the measured flow pulsatility; and
if the difference is less than a first predetermined threshold, generate an alert as the presence of an adverse event.

12. The controller of claim 11, wherein the processing circuitry is further configured to calculate a difference between consecutive points of the moving standard deviation.

13. The controller of claim 12, wherein if the difference between consecutive points of the moving standard deviation is less than a second predetermined threshold, the processing circuitry is further configured to generate the alert as to the presence of the adverse event.

14. The controller of claim 12, wherein the processing circuitry is further configured to display the difference between consecutive points of the moving standard deviation.

15. The controller of claim 11, wherein the processing circuitry is further configured to display the moving standard deviation in the log file.

16. The controller of claim 11, wherein the processing circuitry is further configured to display the moving mean in the log file.

17. The controller of claim 11, wherein the processing circuitry is further configured to display the alert in the log file.

18. The controller of claim 11, wherein the first predetermined threshold is less than 1000.

19. The controller of claim 11, wherein the second predetermined threshold is −180.

20. A method of determining adverse events from log file data from operation of an implantable blood pump, comprising:
measuring a flow pulsatility from flow rate data recorded in a log file;
calculating a moving mean of the measured flow pulsatility;
calculating a moving standard deviation of the measured flow pulsatility;
calculating a difference between the moving mean of the measured pulsatility and the moving standard deviation of the measured flow pulsatility;
calculating a difference between consecutive points of the moving standard deviation;
if the difference is less than a first predetermined threshold, generating an alert as the presence of an adverse event; and
if the difference between consecutive points of the moving standard deviation is less than a second predetermined threshold, generating the alert as to the presence of the adverse event.
